(19) United States
(12) Patent Application Publication
(10) Pub. No.: US 2007/0122449 A1
(43) Pub. Date: May 31, 2007

(54) Biodegradable implantable drug delivery device

(76) Inventors: Yahya E. Choonara, Parktown (ZA); Michael P. Danckwerts, Parktown (ZA); Viness Pillay, Parktown (ZA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/288,035

(22) Filed: Nov. 28, 2005

(51) Int. Cl. A61F 2/02

(52) U.S. Cl. 424/426

(57) ABSTRACT

The invention relates to biodegradable and implantable devices for the in situ delivery of a pharmaceutical composition to a human or animal that comprise a generally discoid body, having at least one aperture through which the body is anchorable by suture, and that are formed from biodegradable polymeric compositions. The invention relates to methods of manufacturing devices for the in situ delivery of a pharmaceutical composition to a human or animal that comprise a generally discoid body, having at least one aperture through which the body is anchorable by suture, and that are formed from biodegradable polymeric compositions. The invention relates to a therapeutic method that comprises inserting a biodegradable and implantable device that comprises a generally discoid body, having at least one aperture through which the body is anchorable by suture, and that is formed from a biodegradable polymeric composition, into a body cavity of a human or animal.

### FIELD OF THE INVENTION

This invention relates to a biodegradable, implantable device for the in situ delivery of a pharmaceutical composition and to a method of manufacturing such a device.

### BACKGROUND OF THE INVENTION

The development of soluble or biodegradable polymers has revolutionized the administration of sustained release formulations or dosage forms of pharmaceutical compositions in treating humans or animals. Briefly, a polymer having a desired biodegradability coefficient for a particular region of the human or animal body is mixed with a pharmaceutical composition and formed, usually by an extrusion process, into a delivery form which is an elongate, oval or circular tablet or disk. When implanted in a human or animal body or ingested, the polymer biodegrades or dissolves slowly depending on its biodegradability coefficient. This results in a sustained release of the pharmaceutical over a predetermined period of time.

The above-described drug delivery device works well for orally ingestable pharmaceuticals. There are, however, situations where the pharmaceuticals are applied to another part of the body and the period over which the pharmaceuticals are released is measured in months rather than minutes or hours. An example of such an application is contraceptive pellet which is injected into the gluteus maximus muscle and releases an oestrogen or similar compound over a period of between three and six months.

The above-described implant is suitable for use in body regions where the pellet can be retained in place by surrounding tissue. There are, however, situations where a drug must be administered over a period of time in an area where some form of securement of the pellet is necessary. Such regions include the intraocular regions, within blood or lymphatic vessels and intraperitoneally.

An intraocular implant known to the applicant is the Vitraserts® implant which is produced by Chiron Vision Incorporated of the United States of America. This implant consists of a pellet of a semi-permeable polymer containing a drug to be delivered to the eye. The pellet is attached to a suturing strip made of a strip of polyvinyl alcohol which is not biodegradable. The suturing strip has a bore therethrough for suturing the implant in the intraocular space. The disadvantage of such an implant is that a second surgical procedure is required to remove the non-biodegradable portion of the implant after the drug supply is exhausted. This doubles the risk of infection and possible damage to the eye.

In this specification the terms “biodegradability coefficient” when used to refer to a biodegradable polymer is to be interpreted as providing a rough indication of the speed with which the polymer will degrade in the human or animal body. Thus, a polymer with a high biodegradability coefficient will take a longer time to biodegrade than a polymer with a low biodegradability coefficient situated in the same region of the body.

### OBJECT OF THE INVENTION

It is an object of this invention to provide a biodegradable and implantable device for the sustained in situ delivery of a pharmaceutical composition to the human or animal body, to provide a method of manufacturing such a device and to provide a method of treating the human or animal body using said device.

### SUMMARY OF THE INVENTION

In accordance with this invention there is provided a biodegradable and implantable device for the in situ delivery of a desired pharmaceutical composition to a human or animal body said device comprising a generally discoid body formed from a biodegradable polymeric composition, the body having at least one aperture therethrough whereby said body is anchorable, in use, within a human or animal body by a suture.

There is also provided for the body to contain a desired pharmaceutical composition and for said body to release the pharmaceutical composition as the body biodegrades.

There is further provided for the body to have a single, alternatively a plurality, preferably two or three, of apertures therethrough and, in addition to said aperture or apertures, for the circumferential periphery of the body to have a number of surface area increasing slots.

There is also provided for the body to have biodegradable polymeric sheets on each of the planar surfaces thereof, the planar polymeric sheets having a greater biodegradability coefficient than the polymer forming the body as thus, in use, permitting the body forming polymer to biodegrade before the sheets.

There is further provided for the aperture, apertures or slots to be shaped such that, when the body forming polymer degrades, the surface area of the biodegradable portion of the body remains relatively constant.

The invention also provides for a method of manufacturing a device for the sustained, in situ, delivery of a desired pharmaceutical composition to a human or animal body, said method comprising the steps of:
a. preparing a biodegradable polymeric composition having a biodegradability coefficient suitable for sustained release of a desired pharmaceutical composition in a particular region of a human or animal body;
b. mixing a desired pharmaceutical composition with the biodegradable polymeric composition; and
c. forming an implantable body, having a generally discoid shape and having at least one aperture therethrough for suturing said body in place in a human or animal body, in a tablet press using a suitably formed die to form said aperture or apertures.

There is also provided for the method to include forming a plurality, preferably two or three apertures through the body and, optionally, a number of surface area increasing slots in the circumferential periphery of the body.

There is also provided for the method to include applying biodegradable polymer sheets to each of the generally planar surfaces of the body, the sheets having a higher biodegradability coefficient than the body forming polymer thus permitting the body to degrade, in use, before the sheets.

The invention also extends to a method of treating a human or animal comprising inserting a device as described above into a body cavity of said human or animal and suturing the body in place to prevent movement of the body within said body cavity.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

Embodiments of the invention will be described below by way of example only and with reference to the accompanying drawings in which:

FIG. 1 is a schematic side perspective view of a first embodiment of device for the sustained, in situ, delivery of a pharmaceutical composition to a human or animal body;

FIG. 2 is a second embodiment of a device for the sustained, in situ, delivery of a pharmaceutical composition to a human or animal body;

FIG. 3 is a third embodiment of a device for the sustained, in situ, delivery of a pharmaceutical composition to a human or animal body;

FIG. 4 is a fourth embodiment of a device for the sustained, in situ, delivery of a pharmaceutical composition to a human or animal body;

FIG. 5 is a cross-sectional side view of the embodiment of a device for the sustained, in situ, delivery of a pharmaceutical composition to a human or animal body of FIG. 1;

FIG. 6 is a cross-sectional side view of the embodiment of a device for the sustained, in situ, delivery of a pharmaceutical composition to a human or animal body of FIG. 2;

FIG. 7 is a schematic side view of the device for the sustained, in situ, delivery of a pharmaceutical composition to a human or animal body of FIG. 1 sutured within an intraocular cavity;

FIG. 8 is a schematic side view of a fifth embodiment of a device for the sustained, in situ, delivery of a pharmaceutical composition to a human or animal body sutured within an artery;

FIG. 9 is a schematic drawing of the tooling used to produce a, experimental device for the sustained, in situ, delivery of a pharmaceutical composition to the human or animal body;

FIG. 10 is a schematic diagram of the production sequence of the experimental devices of FIG. 9;

FIG. 11 shows force displacement profiles generated for the calculation of the Brinell Hardness Number (BHN) values for the experimental devices of FIGS. 9 and 10;

FIG. 12 shows compressibility profiles for various polymers investigated;

FIG. 13 shows mass loss profiles of the experimental devices using various grades of PLGA as a function of time;

FIG. 14 shows typical scanning electron micrographs of erosion of the experimental devices in SVH over a 24 week period;

FIG. 15 shows typical scanning electron micrographs of surface erosion of the experimental devices in SVH over a 24 week period;

FIG. 16 shows the effect of foscarnet and ganciclovir loading on the in vitro release from the Resomer® RG504 experimental device; and,

FIG. 17 shows the effect of PLGA viscosity on the in vitro release from the experimental device at a constant bioreactive load.

### DETAILED DESCRIPTION OF THE DRAWINGS

Referring to FIGS. 1 to 4, a biodegradable and implantable device (1) for the in situ release of a desired pharmaceutical composition to a human or animal body (not shown) comprises a generally discoid body (2) having means (3) whereby said body (2) is anchorable, in use, within a human or animal body (not shown) by at least one suture (not shown).

The discoid body (2) is formed from a biodegradable polymeric composition which is selected from a range of similar compositions according to the locality in the human or animal body in which the device (1) is to be inserted or implanted and the period over which the pharmaceutical composition is to be released which can vary from one or two hours to several months.

The device (1) is made by mixing a pharmaceutical composition with a suitable biodegradable polymer and then forming the device (1) using a pill or tablet press fitted with a suitably shaped die which forms the means (3) whereby the body (2) of the device (1) is sutured into place.

Referring specifically to FIG. 1, the means (3) for suturing the body (2) in place is in the form of a generally circular aperture extending through the body (2). In use, the body (2) is located within a cavity or space in the human or animal body and a suture or stitch is threaded through the aperture and secured to body tissue. The device (1) is thus kept in position and cannot migrate through the body to an untargeted organ or to block a blood or lymphatic vessel or, where the device is implanted intraocularly, affect vision.

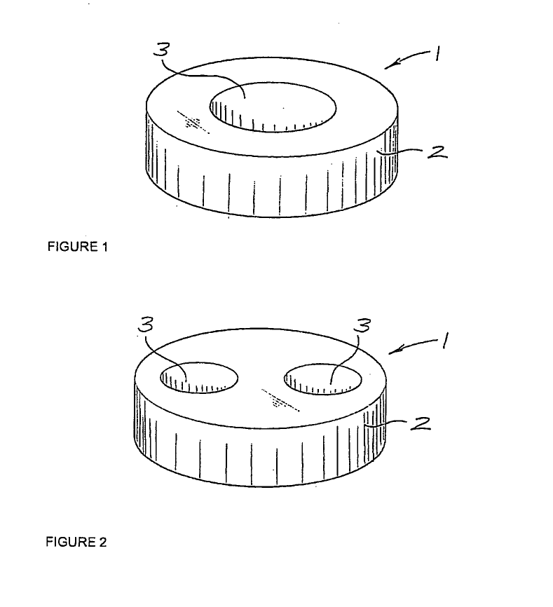

Referring to FIG. 2, the means for suturing the body (2) in place is a pair of apertures (3) extending through the body (2). It is envisaged that a single aperture (3) can be used where the device (1) is to be implanted in a region of the human or animal body where the device (1) will not be subjected to strong forces. One such region is within the eye. Alternatively and where the device (1) will be subjected to strong forces such as in a blood vessel, particularly an artery, or the alimentary canal, both apertures (3) can be employed to suture the device (1) in position.

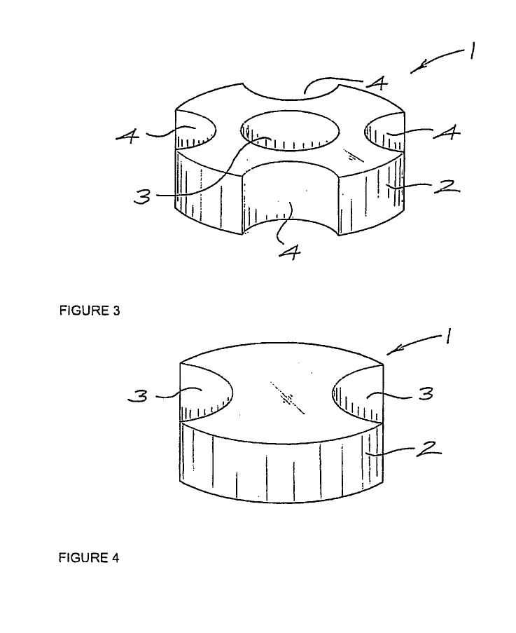

Referring especially to FIG. 3, the device (1) has a single, central aperture (3) through which a suture can be threaded. In addition, the device (1) has four circumferentially spaced, accurate slots (4). It is envisaged that while the slots (4) can also be used to facilitate securement of a suture they will also increase the biodegradable surface area of the device (1) thus enabling the rate of release of a desired pharmaceutical to be controlled within relatively narrow parameters.

Referring now to FIG. 4, the device (1) illustrated here does not have an 20 aperture. In this embodiment the suture is secured to the device (1) by looping it around a pair of diametrically opposed accurate slots (3).

In general, it is envisaged that the release of a pharmaceutical composition mixed with the biodegradable polymer is directly proportioned to the surface area of polymer and pharmaceutical exposed. It is also envisaged that as the circumferential periphery of the device reduces in size and, consequently, surface area, the inner wall of the aperture or apertures and, where present, arcuate slots, increases in size and, consequently, in surface area. By correctly dimensioning the radii of the aperture or apertures and slots it is envisaged that the surface area of the device will remain relatively consistent as the polymer degrades and that the release of the pharmaceutical composition will be consistent.

In addition, by correctly dimensioning and positioning the aperture or apertures and slots it is envisaged that a variable release of the pharmaceutical composition can be achieved. Thus, for example, if a plurality of relatively shallow slots are formed in the periphery of the device the initial release of pharmaceutical will be greater than when the polymer has degraded such that the slots disappeared. Conversely, if a plurality of small apertures are formed through the device then as the polymer degrades the surface area will increase and a greater concentration of pharmaceutical will be released towards the end of the devices lifetime.

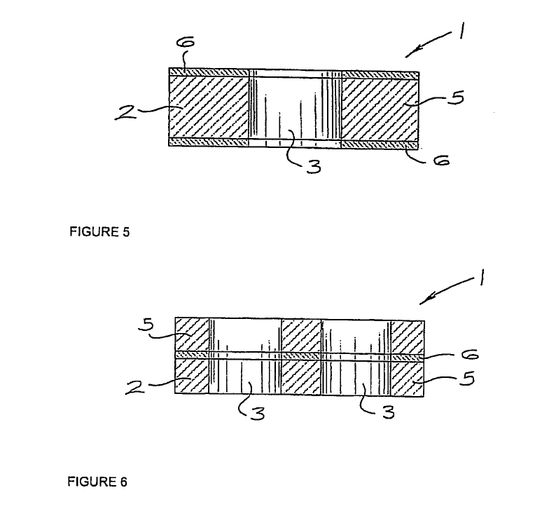

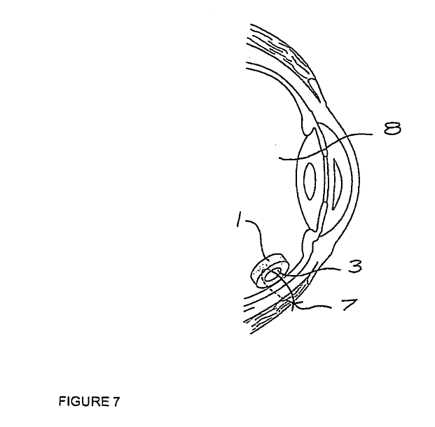

Referring now to FIGS. 5 and 6, devices (1) for the sustained release of more than one pharmaceutical composition into a human or animal body are illustrated. In general this involves utilising a primary biodegradable polymer and pharmaceutical mixture (5) from which the main body of the device (1) is formed together with a secondary biodegradable polymer and pharmaceutical mixture (6) which is formed as a lamination with the primary mixture (5) in the device (1). Depending on the desired pharmaceutical release kinetics, a biodegradable polymer having a suitable biodegradability coefficient is selected.

Accordingly, if it is desired to have an initial, fast release of a particular pharmaceutical followed by a sustained, slower release of another pharmaceutical or indeed of the same pharmaceutical, a polymer having a low biodegradability coefficient is selected as the secondary polymer (6) and it is laminated as shown in FIG. 5. Conversely, if a sustained release over a very long period of time is required then a secondary polymer (6) having a high biodegradability coefficient is sandwiched between the primary polymer (5) as shown in FIG. 6.

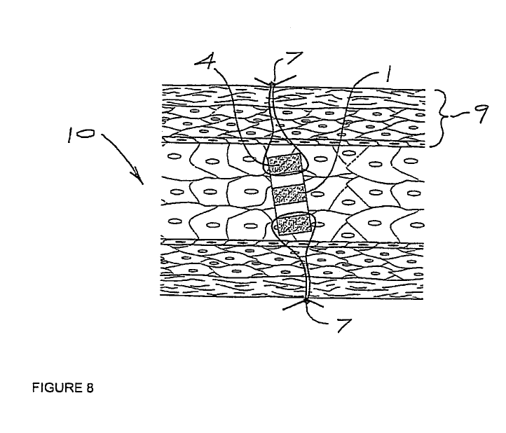

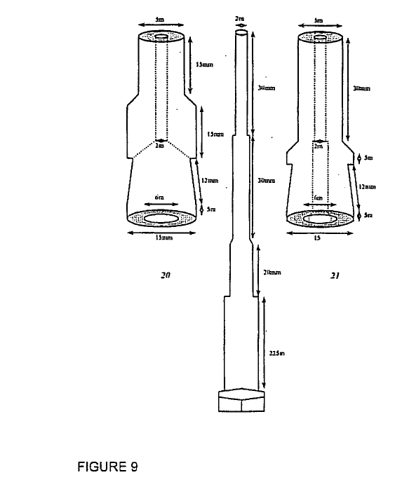

FIGS. 7 and 8 illustrate the applications of devices (1) according to different embodiments of the invention to the human or animal body. In FIG. 6 a device (1) having a single, central aperture (3) is shown in an intraocular application where the device (1) is secured by a single suture (7) to the eye (8).

In FIG. 8, a device (1) having two apertures and four circumferential slots (4) is shown secured by two sutures (7) to the walls (9) of an artery (10). In this application it is envisaged that the number of apertures and slots will also enable blood to flow through the artery (10) with only a minimal reduction in the artery's cross-sectional area.

Exemplification

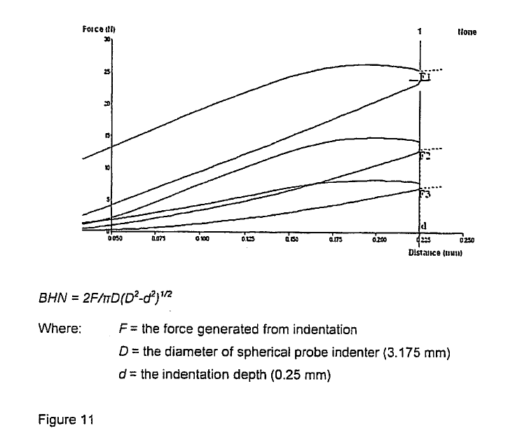

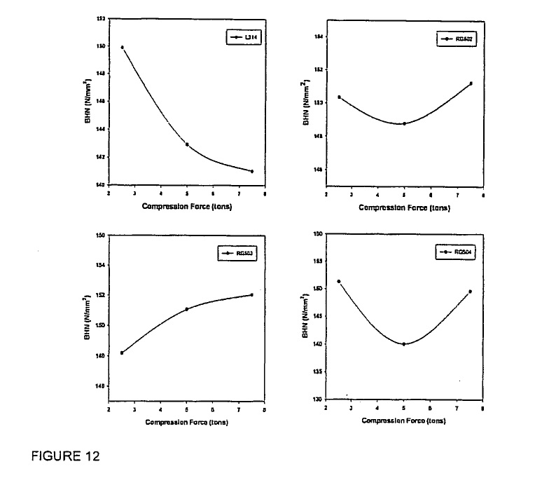

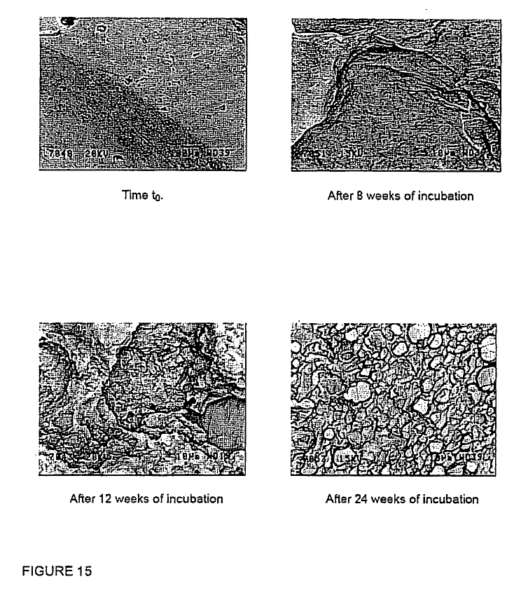

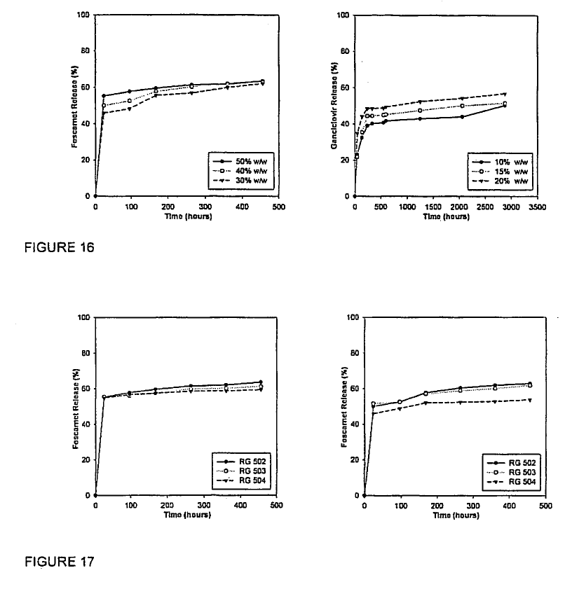

Embodiments of the invention will be described below by way of the exemplification within and with reference to FIGS. 9 to 17. A biodegradable, intraocular implantable device for use in treating cytomegalovirus infections is described as well as a method of manufacturing the device and a method of using it together with biodegradability profiles.

1. Materials and Methods

Resomer® grades RG502, RG503, RG504, and RG756 consisting of poly(lactideco-glycolide) (PLGA) with a 50% lactide content, were purchased from Boehringer Ingelheim (Ingelheim, Germany). These polymers are all biodegradable and have inherent viscosities ranging from 0.16-8.2 dl/g. All polymers were ground in a mortar and the fraction passing through a sieve with an aperture size of 125 μm (Endcotts Test Sieves, Ltd., London, United Kingdom) was used. Phosphonoformic acid (Foscarnet) was obtained from Sigma-Aldrich Co. (Aldrich, Germany). Ganciclovir was purchased from Roche Products (Pty) Ltd. (Isando, South Africa). The antiviral bioactives were used as supplied.

1.1 Preparation of the Device

Referring to FIG. 9, a specially designed punch set (1) was provided by P.A. Cuthbert & Co. (Pty) Ltd. (Johannesburg, South Africa) and engineered by Holland Tableting Science (London, United Kingdom). The punch set consisted of a lower (20) and upper punch (21), a die (not shown), and central-rod (22).

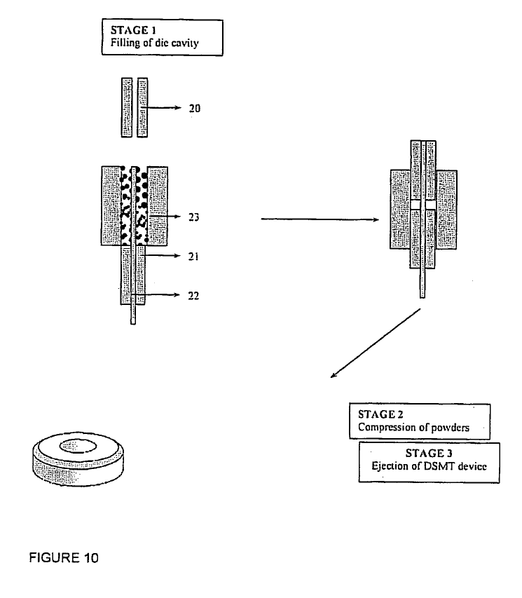

Referring specifically to FIG. 10, both the upper and lower punches (20 & 21) contained a longitudinal central hole for the insertion of the rod (22), which enabled a doughnut-shaped tablet to be compressed around it. The specifications of the punch set are listed in Table 1.

TABLE 1
Specifications of the tooling used to compress the device

| Tooling | Diameter (mm) | Inner Hole (mm) | Length (mm) |
|---|---|---|---|
| Upper punch | 5.00 | 2.00 | 47.00 |
| Lower punch | 5.00 | 2.00 | 52.00 |
| Central rod | 2.00 | N/A | 305.00 |
| Die | 40.00 | 6.00 | 20.00 |

All blending of drug and polymer was performed using an Erweka AR 400 Cube Blender (Erweka, Germany), and a Manesty F3 eccentric (single punch) tableting press (Manesty Machines, United Kingdom) was used for directly compressing the powder blends into the doughnut-shaped mini-tablet (DSMT) device. A model TR 104 mass balance supplied by the Denver Instrument Company (Denver, USA.), was used for all gravimetrical analysis.

The directly compressed formulations consisted of two principal components, namely, the antiviral bioactives and the various biodegradable polymers. With the appropriate choice of polymer, compression of the mixture led to the formation of a matrix tablet. To study the influence of different variables on the release rate of the device, formulations containing various concentrations of bioactive material were produced, and at the same time, the polymer grades were varied to produce different implants.

Devices were produced consisting of 30% w/w, 40% w/w, 50% w/w, 60% w/w, and 70% w/w foscarnet and 10% w/w, 15% W/w, and 20% w/w ganciclovir, in each of the three Resomerg grades chosen in fabricating the device. These concentrations were selected on the basis of the dose required for each bioactive in treating cytomegalovirus retinitis (CMV-R) over a sustained period of time. The various formulations were individually pre-weighed and blended for 10 minutes and then manually fed into the die cavity. The press was set to compress the devices to a thickness of 2 mm.

The variation in compression forces were accomplished by firstly adjusting the upper punch to its lowest position in order to ensure a maximum stable compression force exerted from above, while adjusting the lower punch accordingly by elevating the punch for higher compression forces, or alternatively lowering it for lower compression forces. The adjustments on the lower punch were executed using inherent calibration marks positioned on the adjustment bearing of the tableting press. The flywheel of the tablet press was then twisted backward as far as possible (to the limit where the lower punch started to rise again). This was done to ensure that the tablet press would have enough time to speed up to a relatively constant velocity during compression. The powder blends were then compressed at room temperature. FIG. 3 illustrates a schematic diagram of the production sequence.

1.2 Textural Analysis

Textural analysis was employed to characterize the compressibility of the polymers used in fabricating the DSMT device, employing the TA.XTplus Texture Analyzer (Stable Micro Systems, England) fitted with a probe having a spherical indenter of 3.125 mm in diameter and a 5 kg load cell. The indentation hardness was represented by a conversion to the Brinell Hardness Number (BHN). The textural settings used to calculate the BHN values are depicted in Table 2 where:

TABLE 2
Textural settings employed for BHN value calculations

| Test Parameters | Settings | |
|---|---|---|
| Pre-test speed | 1.000 | mm/sec |
| Test speed | 0.500 | mm/sec |
| Post-test speed | 1.000 | mm/sec |
| Compressive distance | 0.250 | mm |
| Trigger force | 0.050 | N |

1.3 Erosion Studies

Mass loss of the DSMT matrix was evaluated by gravimetrical analysis. Individual DSMT devices were initially weighed, after which degradation was allowed to progress in 4 ml of SVH (pH 7.4, 37′C) in an oscillating laboratory incubator (Labcon® FSIE-SPO 8-35, California, USA) set to oscillate at 50 r.p.m. At predetermined intervals the DSMTs were removed from the vials incubated and gently rinsed with deionised water to remove any superficial particles resulting from undissolved buffer salts. The hydrated matrices were then vacuum-dried and weighed accordingly to assess any gravimetric changes. All experiments were performed in triplicate. The initial and final masses were used to calculate the percentage mass loss (ML %) using the following equation:
ML %=100(Wₒ Wᵣ/Wₒ
Where: Wₒ=initial mass of the device Wᵣ =residual mass of the same dried and partially eroded device.

1.4 Scanning Electron Microscopy

Samples were prepared for photomicrographs by applying a thin layer of colloidal graphite on aluminum stubs and mounting the DSMT devices on the graphite to hold them in place during microscopic examination. The devices were then coated with a thin layer of gold-platinum using a sputter coater under an electrical potential of 15 kV. Several photomicrographs were produced by scanning fields, selected at different magnifications using a Jeol JSM-840 scanning electron microscope (Tokyo, Japan).

1.5 In Vitro Drug Release Studies

A modified closed-compartment USP XXV dissolution testing apparatus was utilized in performing all the release studies. At time zero, the DSMT devices were immersed in 4 ml of SVH, (pH 7.4, 37° C.) in closed vials and placed in an oscillating laboratory incubator (Labcon© FSIE-SPO 8-35, California, USA) set to at 50 r.p.m. At predetermined intervals, 2 ml of the release medium was sampled and 2 ml of fresh SVH was replaced to the sampled vial to maintain sink conditions.

1.6 Analysis of the Antiviral Bioactives

Analyses of foscarnet and ganciclovir were performed on a System Gold (Beckman, San Ramon, Calif.) high performance liquid chromatograph (HPLC) equipped with a 20 μl loop, and array detector. The system was fitted with a Discovery C₁₈ (particle size 5 pm, 4.6 mm id., 150 mm) analytical column (Supelco, USA). Samples of 20 μl were injected using a microlitre syringe (705 NR 50 μl, Hamilton Bonaduz AG, Switzerland).

The mobile phase for analysing foscarnet samples was composed of 0.005 M sulphuric acid/methanol (95:5% v/v) and approximately 0.09% w/v of tetrahexylammonium hydrogensulphate (THAHSO4) as an ion pairing reagent. A solvent flow rate of 1.5 ml/min was maintained. In the case of ganciclovir, the mobile phase was composed of 0.05 M ammonium acetate (pH 6.5)/acetonitrile (96:4% v/v), and a solvent flow rate of 1.0 ml/min was maintained. Prior to use, the column was equilibrated by passing 45 ml of solvent through the system. The eluant was monitored at an analytical wavelength set at 254 nm. The entire assay procedure was performed at room temperature. Ascorbic acid and acyclovir were used as the respective internal standards for foscarnet and ganciclovir analysis.

2. Results and Discussion

2.1 Compressibility of the PLGA Polymers Employing Textural Analysis

Table 3 lists the force values (N) generated from indentation of the PLGA compacts, as well as the BHN values. The compressibility profiles for each polymer investigated are shown in FIGS. 12a, b, c and d. The indentation hardness of the compacts indicated that the material formed moderately soft compacts, as the compression force increased. Notably, the BHN values for the amorphous regions of the polymer in the DSMT showed significant variability as seen from the compressibility profiles obtained in FIG. 12 for the polymers L214, RG502, RG503 and RG504. This behaviour was contrary to that expected. One observation was that the sudden upward trend for L214 and RG504 of the compressibility profile at a compression force of 7.50 tons could possibly be attributed to an increase in crystallinity of the PLGA compacts as a result of amorphous regions being subjected to deformation earlier than the crystalline regions during application of compression force. Hence this leads to an increase in crystallinity in the remaining polymer and thus a higher BHN value.

TABLE 3
BHN values obtained at the different compression forces

| | Compres- | | |
|---|---|---|---|
| | sion | Indentation | |
| Polymer Type | Force | Force (N)^{t} | BHN |
| (N/mm²) [symbol] | (tons) | Mean ± SD | Mean ± SD |
| Resomer ® RG502 | 2.50 | 26.228 ± 0.216 | 150.33 ± 1.240 |
| Resomer ® RG502 | 5.00 | 25.955 ± 0.302 | 148.77 ± 1.731 |
| Resomer@ RG502 | 7.50 | 26.382 ± 0.375 | 151221 ± 2.148 |
| Resomer ® RG503 | 2.50 | 25.852 ± 0.349 | 148.18 ± 2.006 |
| Resomer ® RG503 | 5.00 | 26.355 ± 0.120 | 151.06 ± 0.689 |
| Resomer ® RG503 | 7.50 | 26.525 ± 0.912 | 152.04 ± 0.523 |
| Resomer ® RG504 | 2.50 | 26.396 ± 1.062 | 151.30 ± 6.089 |
| Resomer ® RG504 | 5.00 | 24.426 ± 1.184 | 140.00 ± 6.785 |
| Resomer ® RG504 | 7.50 | 26.100 ± 0.368 | 149.60 ± 2.107 |
| Resomer ® L214 | 2.50 | 26.152 ± 0.816 | 149.90 ± 4.678 |
| Resomer ® L214 | 5.00 | 24.934 ± 0.235 | 142.92 ± 1.346 |
| Resomer ®L214 | 7.50 | 24.602 ± 0.411 | 141.01 ± 2.356 |

^{t}Force produced on indentation of compacts

This observation highlights the important differences in the mechanical properties of amorphous pharmaceutical material, which is temperature and pressure dependant in their response to applied mechanical stress. Firstly, the copolymer PLGA is an amorphous type material. Amorphous materials do not have melting points but rather go through a glass transition temperature (T9). PLGA 50:50 has a T_{g} value of 39.85° C.

Secondly, poly(lactide) (PLA) is thermally unstable (Zhang et al., 1992). Hence, the presence of this monomer randomly distributed within the copolymer backbone structure tends to increase chain flexibility and therefore, at elevated temperatures during the direct compression technique, it's inclined to contribute to the opening of the polymer structure and widening in the copolymer molecular weight distribution and a decrease in yield pressure. The yield pressure indicates the point where forces involved in consolidation have essentially been ‘broken’.

The yield pressure for PLGA is well below the lowest pressure used in the compressibility of the DSMT's and its value has been significantly surpassed. In most cases the Manesty tabletting press used cannot exceed a compression force of 1200 pounds (1 ton=2000 pounds) when employing PLGA as a directly compressible excipient.

Compression of the PLGA powders generates significant heat within the internal structure of the tablet. The excess heat is dissipated and escapes gradually through the surface of the tablet matrix. Since it would have required significant modification of the current testing equipment to permit data to be collected in order to monitor these in situ elevated temperatures, this phenomenon could not be explored in detail as part of the current work.

At temperatures above the T_{g} value, amorphous material would show a transformation in its behaviour with an increase in ductility. Subsequently what is occurring is that the material is transitioning into a more rubbery state. It is well-known that above the T_{g} value molecular motions are very rapid which may lead to a decrease in the extent of polymer chain entanglement and an increased mobility of the polymer chain segments, whereas below the T_{g} value these motions are restricted and are regarded as almost ‘frozen’.

With regard to the DSMT device, the study commenced employing 1 ton of uniaxial compression force, which has already exceeded the yield pressure (>1200 pounds), resulting in increased heat generation and dissipation, that is well above the stated T_{g} value. This excess heat induces structural changes at a molecular level and causes a deformation of the matrix that leads to stress relief at heterogeneous stress loci distributed within the tablet matrix. Consequently stress relaxation and surface disruption occur on a micro-level. This is consistent with the broader expectation that amorphous type materials have a higher free energy. Essentially, the greater the compression force, more heat is generated when above the yield value, which results In exceeding the T_{g} value. The DSMT consequently turns into a more rubbery state and therefore the higher compression force produces a ‘softer’ matrix

2.2 Erodibility of the DSMT Device

Table 4 lists the residual masses obtained for the DSMT devices investigated. The effect of the various polymer grades used in fabricating the DSMTs on mass loss profiles are shown in FIG. 6. The shapes of the curves were found to be similar for all the investigated polymers. As seen from FIG. 6, the composition of the polymers employed in fabricating the DSMTs have a significant effect on the mass loss profile, or otherwise the erosion characteristics.

TABLE 4
Residual masses and % mass loss at various time intervals

| | Residual Masses | |
|---|---|---|
| Polymer Type and Time | (mg) ± SD | % Mass Loss^{t} |
| Resomer © RG502 | | |
| At 8 weeks | 49.75 ± 5.05 | 48.35 |
| At 12 weeks | 46.75 ± 8.18 | 51.47 |
| At 24 weeks | 44.90 ± 5.98 | 53.39 |
| Resomer ® RG503 | | |
| At 8 weeks | 55.23 ± 5.01 | 42.09 |
| At 12 weeks | 49.65 ± 8.41 | 47.94 |
| At 24 weeks | 46.25 ± 5.59 | 51.50 |
| Resomer © RG504 | | |
| At 8 weeks | 61.70 ± 4.10 | 35.53 |
| At 12 weeks | 53.60 ± 8 06 | 43.99 |
| At 24 weeks | 49.50 ± 6.93 | 48.37 |

^{t}calculated using the % mass loss equation

3.3 Scanning Electron Microscopy

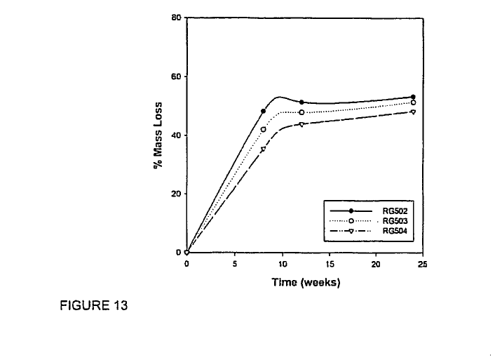

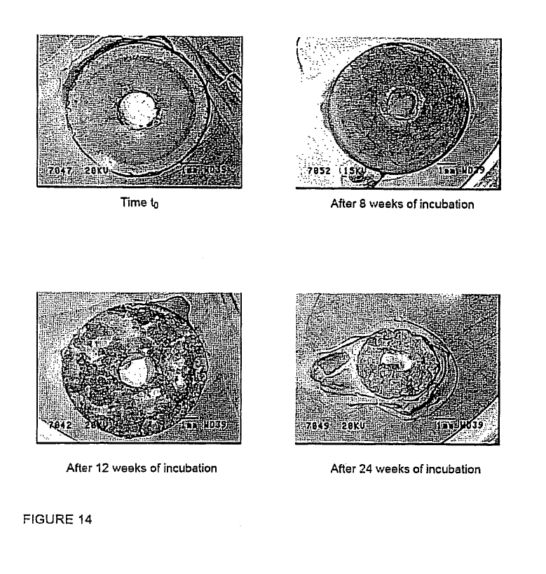

The morphological characteristics of the DSMT devices undergoing erosion at various predetermined time intervals are shown in FIGS. 13 and 14. The photomicrographs were taken of a sample used for determining gravimetric changes as described above.

Before storage in SVH at 37° C., the surface of the DSMT device appeared smooth without many cracks and pores visible within the matrix (FIG. 7a, and 8a). After 8 weeks of storage (FIG. 7b, and 8b) the DSMT surface was still fairly smooth, but a few superficial cracks emerged. These cracks were probably due to the drying process during SEM sample preparation. Furthermore, it did not show any channel-like structures, not allowing much diffusion of dissolution medium into the device matrix. At 12 weeks, (FIG. 7c and 8c) surface pores and cracks were prominently engulfing the matrix, resulting in channel-like structures that permit easy diffusion of release medium into the drug delivery system. At this stage the DSMT device began to evidently show signs of size reduction. This can be noticeably seen in FIGS. 7d and 8d by the outstanding porous surface with a vast amount of cracks and channel-like structures appearing after 24 weeks of incubation. During observation, the cracks vaguely increased, indicating a looser structure of the matrix as the device continued to erode.

2.4 In Vitro Release Studies

In general, the release profiles of both antiviral bioactives from the biodegradable polymer matrices have a biphasic release pattern: an initial burst and a second phase that is derived from difflusional release before the onset of polymer erosion. The initial burst may have resulted from the rapid release of the drugs deposited on the surface of the matrix. During the difflusion phase, the bioactives were released slowly, and possibly was controlled by the degradation rate of the polymer and the drug loading. The rate of release in the diffusion phase tended to be more prolonged in the DSMT's consisting of the polymer with the higher inherent viscosities. An increase in the drug load of the device may also increase the release rate and an overload of drug may result in the release of a large amount of drug as an initial burst.

The release kinetics during the second phase was fairly consistent at the various drug loading concentrations. The devices have a biphasic pattern and in the higher viscosity polymer, the duration of drug release tends to be more prolonged (FIG. 9 and 10).

3. Conclusion

PLGA can be regarded as suitably compressible for designing implantable devices such as the DSMT using relatively low compression forces which aid in trouble-free manufacturing with regard to wear on major tableting equipment such as punches and dies. The veracity of the compact structure of the DSMT device was retained even after 24 weeks of Incubation during the erosion studies, indicating that the device is suitable as a biodegradable drug delivery system.

The DSMT device generally has a biphasic release pattern: an initial burst and a diffusional phase. The inherent viscosity of the polymers employed and the drug loading characteristics affects the duration and rate of bioactive release. These factors are able to regulate the release profile from the DSMT device in order to achieve the desired release kinetics of the drug delivery system. The DSMT with a superior load of bioactive had a larger quantity of bioactive released during the initial phase.

The DSMT has proved to be a flexible and versatile biodegradable intraocular drug delivery system that provides rate-modulated release of antiviral bioactives to the posterior segment of the eye and may be suitable for the treatment of CMV-R.

While this invention has been particularly shown and described with references to preferred embodiments thereof, it will be understood by those skilled in the art that various changes in form and details may be made therein without departing from the scope of the invention encompassed by the appended claims.

What is claimed is:

1. A biodegradable and implantable device for the in situ delivery of a desired pharmaceutical composition to a human or animal body said device comprising a generally discoid body formed from a biodegradable polymeric composition, the body having at least one aperture therethrough whereby said body is anchorable, in use, within a human or animal body by a suture.

2. A biodegradable and implantable device for the in situ delivery of a desired pharmaceutical composition to a human or animal body as claimed in claim 1 in which the body contains a desired pharmaceutical composition.

3. A biodegradable and implantable device for the in situ delivery of a desired pharmaceutical composition to a human or animal body as claimed in claim 2 in which the body to releases the pharmaceutical composition as the body biodegrades.

4. A biodegradable and implantable device for the in situ delivery of a desired pharmaceutical composition to a human or animal body as claimed in claim 3 in which the body has a single aperture therethrough.

5. A biodegradable and implantable device for the in situ delivery of a desired pharmaceutical composition to a human or animal body as claimed in claim 3 in which the body has a plurality of apertures therethrough.

6. A biodegradable and implantable device for the in situ delivery of a desired pharmaceutical composition to a human or animal body as claimed in claim 5 in which the body has two apertures therethrough.

7. A biodegradable and implantable device for the in situ delivery of a desired pharmaceutical composition to a human or animal body as claimed in claim 5 in which the body has three apertures therethrough.

8. A biodegradable and implantable device for the in situ delivery of a desired pharmaceutical composition to a human or animal body as claimed in claim 1 in which the circumferential periphery of the body has a number of surface area increasing slots.

9. A biodegradable and implantable device for the in situ delivery of a desired pharmaceutical composition to a human or animal body as claimed in claim 1 in which the body has biodegradable polymeric sheets on each of the planar surfaces thereof, the planar polymeric sheets having a greater biodegradability coefficient than the polymer forming the body as thus, in use, permitting the body forming polymer to biodegrade before the sheets.

10. A biodegradable and implantable device for the in situ delivery of a desired pharmaceutical composition to a human or animal body as claimed in claim 8 in which the aperture, apertures or slots in the body are shaped such that, when the body forming polymer degrades, the surface area of the biodegradable portion of the body remains relatively constant.

11. A method of manufacturing a device for the, in situ, delivery of a desired pharmaceutical composition to a human or animal body, said method comprising the steps of:
(a) preparing a biodegradable polymeric composition having a biodegradability coefficient suitable for sustained release of a desired pharmaceutical composition in a particular region of a human or animal body;
(b) mixing a desired pharmaceutical composition with the biodegradable polymeric composition; and
(c) forming an implantable body, having a generally discoid shape and having at least one aperture therethrough for suturing said body in place in a human or animal body, in a tablet press using a suitably formed die to form said aperture or apertures.

12. A method of manufacturing a device for the, in situ, delivery of a desired pharmaceutical composition to a human or animal body as claimed in claim 11 in which the method to includes forming a plurality of apertures through the body.

13. A method of manufacturing a device for the, in situ, delivery of a desired pharmaceutical composition to a human or animal body as claimed in claim 11 in which two apertures are formed through the body.

14. A method of manufacturing a device for the, in situ, delivery of a desiredpharmaceutical composition to a human or animal body as claimed in claim 11 in which three apertures are formed through the body.

15. A method of manufacturing a device for the, in situ, delivery of a desired pharmaceutical composition to a human or animal body as claimed in claim 11 in which a number of surface area increasing slots are formed in the circumferential periphery of the body.

16. A method of manufacturing a device for the, in situ, delivery of a desired pharmaceutical composition to a human or animal body as claimed in claim 11 in which biodegradable polymer sheets are applied to each of the generally planar surfaces of the body, the sheets having a higher biodegradability coefficient than the body forming polymer thus permitting the body to degrade, in use, before the sheets.

17. A method of treating a human or animal comprising inserting a device as claimed in claim 1 into a body cavity of said human or animal and suturing the body in place to prevent movement of the body within said body cavity.
